(19) United States
(12) Patent Application Publication
(10) Pub. No.: US 2008/0181956 A1
(43) Pub. Date: Jul. 31, 2008

(54) Oil-in-water personal care composition

(75) Inventors: Robert Bao Kim Ha, Liberty Twp, OH (US); Paul Robert Tanner, Lebanon, OH (US); Robert John Elsbrock, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/700,379

(22) Filed: Jan. 31, 2007

(51) Int. Cl. A61K 9/14

(52) U.S. Cl. 424/489; 424/501; 604/289

(57) ABSTRACT

A personal care composition in the form of an oil-in-water emulsion is disclosed that provides improves skin feel. The personal care composition comprises from a silicone elastomer; a silicone fluid; a particulate material having a refractive index of less than about 1.8; a polymeric thickener; and water. The personal care composition may be substantially devoid of opacifying particulate material. A consumer product comprising the personal care composition is also disclosed. The consumer product allows for the application of the personal care composition to the skin or other keratinous tissue.

### FIELD OF THE INVENTION

The present invention relates to personal care compositions having a high percentage of silicone elastomer, silicone fluid, and low refractive index powder. The present invention further relates to consumer products containing the personal care composition.

### BACKGROUND OF THE INVENTION

A variety of personal care compositions are available to provide skin care benefits and to counteract what many consider undesirable “signs of skin aging,” such as fine lines, wrinkles and uneven skin texture. To be most effective, compositions generally must be applied regularly and over an extended period of time. This may be especially important when the product is intended to provide a chronic, or long-term, benefit. To encourage frequent usage, it is important that the product have a desirable feel when applied, and also provide some indication that the product is having its intended effect. There exists a continuing need, therefore, to provide personal care compositions that signal an immediate benefit of skin feel and thus encourage repeated use to provide a long-term benefit.

Silicone elastomers and silicone fluids have been used in consumer products to impart an improved skin feel as opposed to hydrocarbon-based products. Silicone elastomers and silicone fluids are typically described as providing a less heavy or greasy feel compared to hydrocarbon-based products. However, previous compositions have typically had a continuous silicone phase with a discontinuous aqueous phase. These water-in-silicone emulsions often exhibit problems with prolonged stability. Furthermore, silicone continuous phase products present processing complexities because water-in-silicone emulsions are typically sensitive to shear. The shear inherent in a large scale-processing line often may cause destabilization of these emulsions. Silicone continuous phases are further disfavored because processing lines after having conveyed silicone fluids are very difficult to clean without the use of solvents or a large quantity of surfactants.

Particulate material can be added to consumer products for a variety of reasons such as to improve the skin feel of the product. Particulate materials also may provide a immediate visible benefit to the skin by diffusely reflecting light, which provides a matting effect to the skin. However, many particulate materials are added to act as an opacifying agent, which effectively turns the consumer product into make-up and like products. The acute and chronic benefits of personal care compositions having opacifying agents are often lost on the user who only appreciates the masking effect the opacifying agents provide.

Therefore, a need exists for a personal care composition providing the improved skin feel benefits by using silicone elastomers and a high percentage of silicone fluids without the adverse effects of a continuous silicone phase including the problems of stability, processing, and cleaning. A further need exists for the personal care composition to comprise a high percentage of a particulate material having a low refractive index.

### SUMMARY OF THE INVENTION

In light of the needs presented above, the present invention relates to a personal care composition in the form of an oil-in-water emulsion providing improved skin feel through the use silicone elastomers, a high percentage of silicone fluid, and a high percentage of particulate material having a low refractive index. The personal care compositions are stable and have a silky, non-greasy feel. In a particular embodiment, the present invention relates to a personal care composition in the form of a oil-in-water emulsion having a silicone elastomer, a silicone fluid, particulate material having a refractive index of less than about 1.8, a polymeric thickener, and water.

In another embodiments, the present invention relates to a consumer product including an applicator comprising a substrate and a reservoir. The reservoir contains a quantity of a personal care composition.

### BRIEF DESCRIPTION OF THE DRAWINGS

FIG. 1 is a perspective view of one embodiment of a consumer product containing a personal care composition.

FIG. 2 is a perspective view of another embodiment of a consumer product containing a personal care composition.

### DETAILED DESCRIPTION OF THE INVENTION

As used herein, the following terms shall have the meaning specified thereafter:

“Personal care composition” means compositions suitable for topical application on mammalian keratinous tissue.

“Skin care actives,” or “actives,” as used herein, means compounds that, when applied to the skin, provide a benefit or improvement to the skin. It is to be understood that skin care actives are useful not only for application to skin, but also to hair, nails, and other mammalian keratinous tissue.

“Stable” and “stability” mean a composition which is substantially unaltered in chemical state, physical homogeneity, and/or color when the composition is at a temperature of from about 1° C. to about 40° C.

“Keratinous tissue” refers to keratin-containing layers disposed as the outermost protective covering of mammals which includes, but is not limited to, skin, hair, nails, cuticles, etc.

“Applied” or “application” means to spread the composition onto keratinous tissue with one or more fingers and/or an implement, as one would be expected to apply a cream to the facial skin.

“Regulating skin condition” means improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic or acute benefit and may include one or more of the following: reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

“Signs of skin aging,” include, but are not limited to, all outward visibly and tactilely perceptible manifestations, as well as any macro- or micro-effects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Insult-affected keratinous tissue,” means keratinous tissue which exhibits discomfort, irritation, an unpleasant or irregular appearance, and the like, for example after exposure to a physical and/or chemical irritant. Non-limiting examples of insult-affected keratinous tissue include sunburn and other types of burns; rashes, such as diaper rash, shaving rash and allergen-induced rashes; discoloration, such as bleaching, staining or hyperpigmentation; skin having nicks and cuts due to, for example, shaving; dry, chapped or rough skin due to exposure to example wind, cold and/or low humidity, etc. Non-limiting examples of insults include radiation, wind, low humidity, allergens, pollutants, chemical and natural irritants, bodily fluids, bodily waste, excessive moisture, bacteria, fungi, etc.

“Non-volatile” means materials that exhibit a vapor pressure of no more than about 0.2 mm Hg at 25° C. at one atmosphere and/or to materials that have a boiling point at one atmosphere of at least about 300° C. “Volatile,” as used herein, all materials that are not “non-volatile” as defined herein.

“Safe and effective amount” means an amount of a compound or composition sufficient to induce a positive benefit but low enough to avoid serious side effects (i.e., provides a reasonable benefit to risk ratio within the judgment of a skilled artisan).

In all embodiments of the present invention, all percentages are by weight of the total composition, unless specifically stated otherwise. All ratios are weight ratios, unless specifically stated otherwise. All ranges are inclusive and combinable; therefore, every range given throughout this specification will include every narrower range that falls within such broader range as if such narrower ranges were all expressly written herein. The number of significant digits conveys neither a limitation on the indicated amounts nor on the accuracy of the measurements. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

### I. Personal Care Composition

The personal care composition of the present invention may be a skin care, cosmetic, and hair care product. The personal care composition may be used as, for example, a moisturizer, conditioners, anti-aging compound, skin lightening compound, sunscreen, sunless tanner, and combinations thereof. In certain embodiments, the composition is applied to the face, neck, hands, arms, and other typically exposed areas of the body. Alternatively, the composition is applied to insult-affected areas of keratinous tissue.

The compositions of the present invention are useful for improving skin appearance and/or feel. The compositions of the present invention may be useful for regulating skin condition and/or improving skin condition. In certain embodiments, the composition is useful for regulating and/or improving the signs of skin aging. The compositions may provide an essentially immediate (i.e., acute) improvement in skin appearance and/or feel following application. It is believed that the acute improvement may be attained with a single or limited number of applications of the composition. However, the compositions may comprise components that provide a gradual (i.e., chronic) improvement in skin appearance and/or feel. It is believed that the chronic improvement may involve multiple, reoccurring, or periodic applications of the composition. The compositions of the present invention may be incorporated into consumer products. In certain embodiments, the consumer products allow the composition to be applied as a spot treatment over a limited area of the skin.

The compositions of the present invention are in the form of an emulsion. While various emulsion forms are known in the art (e.g., oil-in-water, water-in-oil, water-in-oil-in-water, and oil-in-water-in-oil emulsions), the compositions of the present invention are directed to an emulsion having a continuous aqueous phase and a discontinuous (i.e., internal) oil phase. The oil phase of the present invention comprises a silicone oil (which may alternately called silicone fluids). However, the oil phase may also comprise non-silicone oils such as hydrocarbon oils, esters, ethers, and the like.

The aqueous phase typically comprises water. The composition may comprise about 8% to about 74% of water. Alternatively, the composition may comprise about 35% to about 60% of water. The aqueous phase may comprise components other than water (non-water components), including but not limited to water-soluble moisturizing agents, conditioning agents, anti-microbials, humectants and/or other water-soluble skin care actives, to impart an increased benefit to the keratinous tissue. In one embodiment, the non-water component of the composition comprises a humectant such as glycerin and/or other polyols.

Silicone Elastomers The composition of the present invention comprises at least one silicone elastomer. Silicone elastomers are useful for reducing the tackiness of the composition and for providing a pleasant feel upon application. One non-limiting example of a suitable class of silicone elastomers is crosslinked organopolysiloxane (or siloxane) elastomers, which are generally described in U.S. patent application publication US2003/0049212A1. The crosslinked organopolysiloxane elastomers may be categorized as emulsifying or non-emulsifying. “Emulsifying,” as used herein, means crosslinked organopolysiloxane elastomers having at least one polyoxyalkylene (e.g., polyoxyethylene or polyoxypropylene) or polyglycerin moiety. The polyoxyalkylene (e.g., polyoxyethylene or polyoxypropylene) or polyglycerin moiety may serve as the crosslinker within the elastomer. Exemplary emulsifying crosslinked organopolysiloxane elastomers are described in U.S. Pat. Nos. 5,412,004; 5,837,793, and 5,811,487. “Non-emulsifying” means crosslinked organopolysiloxane elastomers are essentially free of polyoxyalkylene or polyglycerin moieties.

The composition of the present invention may comprise from about 0.5% to about 10% or, alternatively, from about 1% to about 5% of one or more silicone elastomers. The indicated percentages are understood to refer to amount of dry elastomer, as opposed to the total amount of elastomer and solvent, used for example for storage and shipping.

In certain embodiments, the composition of the present invention may comprise from about 0.5% to about 10% or, alternatively, from about 1% to about 5% of one or more non-emulsifying crosslinked siloxane elastomers. In these embodiments, the composition may comprise no to substantially no (i.e., less than 0.01%) emulsifying crosslinked siloxane elastomers. Exemplary non-emulsifying crosslinked siloxane elastomers include the CTFA (Cosmetic, Toiletry, and Fragrance Association International Cosmetic Ingredient Dictionary and Handbook, 11^{th} ed.) designated dimethicone/vinyl dimethicone crosspolymers supplied by a variety of suppliers including Dow Corning™ (DC 9506), General Electric™ (SFE 839), Shin Etsu™ (KSG 15 and 16), and Grant Industries (GRANSIL RPS-NA) and dimethicone/phenyl vinyl dimethicone crosspolymer such as KSG 18 available from Shin Etsu™. Other exemplary non-emulsifying crosslinked siloxane elastomer include the CTFA designated dimethicone crosspolymers including Dow Corning™ (DC 9040, DC 9041, DC 9045).

The composition of the present invention may comprise from about 0.5% to about 10% or, alternatively, from about 1% to about 5% of one or more emulsifying crosslinked siloxane elastomer such as described in U.S. Pat. Nos. 5,412,004; 5,837,793; and 5,811,487. Suitable emulsifying crosslinked organopolysiloxane elastomers include dimethicone/PEG-10 crosspolymers such as KSG 24; dimethicone/PEG-10 crosspolymers such as KSG 21 and KSG 210; PEG-15/lauryl dimethicone crosspolymers such as KSG 31, KG 32, KSG 33, KSG 310, KG 320, KSG 330; PEG-15/lauryl dimethicone crosspolymers and PEG-10/lauryl dimethicone crosspolymers such as KSG 34 and KSG 340; dimethicone/polyglycerine-3 crosspolymers such as KSG-710; and lauryl dimethicone/polyglycerine-3 crosspolymers such as KSG 810, KSG 820, KSG 830, and KSG 840. All KSG materials are available from Shin Etsu™.

Silicone Fluid—The composition of the present invention comprises from about 20% to about 50% of a silicone fluid. In certain embodiments, the oil phase comprises a mixture of volatile silicones and/or non-volatile silicones. The silicone fluid is present in an amount of from about 25% to about 40% by weight. In certain embodiments, the silicone fluid may comprise from about 60% to about 90% volatile silicones.

Suitable volatile silicones include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 9 silicon atoms, preferably from about 4 to about 6 silicon atoms. Exemplary cyclic volatile silicones of varying viscosities include Dow Corning DC 244, DC 245, DC 246, DC 344, and DC 345; GE Silicones-OSi Specialties Volatile Silicone 7207 and Volatile Silicone 7158; and GE Silicones SF1202. Suitable linear silicones include the polydimethylsiloxanes containing an average of from about 3 to about 9 silicon atoms. Exemplary linear volatile silicones include Dow Corning DC 200 and DC225 and the Shin Etsu DM-Fluid series. In certain embodiments, the linear volatile silicones generally have viscosities of less than or equal to about 6 centistokes at 25° C., and the cyclic materials generally have viscosities of less than about 10 centistokes at 25° C.

The nonvolatile silicones will have vapor pressures as previously defined. The nonvolatile silicones may exhibit an average viscosity of from about 10 to about 100,000 cps at 25° C. or from about 50 to about 10,000 cps. Examples of suitable nonvolatile silicones include, but are not limited polyalkylsiloxanes, polyarylsiloxanes, polyalkylarylsiloxanes, polyethersiloxane copolymers, and mixtures thereof. Examples of useful oils include the General Electric Viscasil™ series, General Electric SF1075, high viscosity members of the Dow Corning DC 200 series, and Dow Corning DC 556 Cosmetic Grade Fluid.

The personal care composition may also comprise non-silicone oils. Suitable non-silicone oils include nonpolar, volatile hydrocarbon oils are discussed in Cosmetics, Science, and Technology, Vol. 1, 27-104 edited by Balsam and Sagarin, 1972. The nonpolar, volatile hydrocarbon oils may be saturated, unsaturated, aliphatic (straight or branched chains), alicyclic, or aromatic. Examples of preferred nonpolar, volatile hydrocarbon oils include, but are not limited to, isododecane (e.g., Permethyl-99A which is available from Presperse Inc.), isodecane, and the C7-C8 through C12-C15 isoparaffins (e.g., Isopar Series available from Exxon Chemicals).

Other suitable non-silicone oils include polar, nonvolatile oils such as fatty alcohols; fatty acids; esters of mono and dibasic carboxylic acids with mono and polyhydric alcohols; polyoxyethylenes; polyoxypropylenes; mixtures of polyoxyethylene and polyoxypropylene ethers of fatty alcohols; and mixtures thereof. The aforementioned oils may be saturated, unsaturated, aliphatic (straight or branched chains), alicyclic, or aromatic.

Other suitable non-silicone oils include nonpolar, nonvolatile hydrocarbon oils such as paraffinic hydrocarbon oils including mineral oils and certain branched-chain hydrocarbons as discussed in U.S. Pat. No. 5,019,375. Exemplary branched-chain hydrocarbons include Permethyl 103A (average of about 24 carbon atoms), Permethyl 104A (average of about 68 carbon atoms), and Permethyl 102A (average of about 20 carbon atoms); all may be purchased from Presperse, Inc., Somerset, N.J.

Particulate Material—The compositions of the present invention may comprise a first particulate material having a refractive index of less than about 1.8. In other embodiments, the particulate material may have a refractive index of about 1.3 to about 1.7. In other embodiments, the refractive index of the particulate material may be in the range from about 1.3 to about 1.6 as this range closely matches the refractive index of skin. The particulate material may be dispersed and suspended in the composition. Refractive index can be determined by conventional methods.

The particulate material may exhibit a median particle size of from about 0.1 μm to about 40 μm; from about 1 μm to about 20 μm; or, alternatively, from about 2 μm to about 10 μm. Median particle size can be determined by any suitable method known in the art, such as by using coulter-counter equipment or the ASTM Designation E20-85 “Standard Practice for Particle Size Analysis of Particulate Substances in the Range of 0.2 to 75 Micrometers by Optical Microscopy”, ASTM Volume 14.02, 1993, incorporated herein by reference. The median particle size is measured when the particulate material is in the neat form (i.e., in the essentially pure, powder form prior to combination with the carrier of the invention).

The compositions of the present invention preferably comprise from about 5% to about 30% of the particulate material. Alternatively, the compositions of the present invention preferably comprise from about 6% to about 15% or from about 8% to about 14% of the particulate material.

The particulate materials may be inorganic or organic. Typically, the particulate materials are free-flowing and solid (i.e., the particles are not hollow). However, the shape of the particulate material is not limited so long as the particulate material exhibits a low refractive index. Suitable organic particulate materials include those made of polymethylsilsesquioxane (e.g., Tospearl™ 145A available from GE Toshiba Silicone Co., Ltd.), polyamide (e.g., nylon-12 and Orgasol™ 2002D Nat C05 available from Atofina), polyolefines (e.g., Microthene™ FN510-00 available from Equistar), polyacrylates (e.g., ethylene acrylate copolymer, sold under the name FloBead™ EA209 available from Kobo), polymethacrylates (PMMA) (e.g., Micropearl™ M 100 available from Seppic), polystyrene (e.g., Dynospheres™ available from Dyno Particles), polytetrafluoroethylene (PTFE), polyurethanes, starch and starch derivatives, composite particles, and mixtures thereof. Copolymers derived from monomers of the aforementioned materials can also be used. The aforementioned polymers derived from carboxylic acid containing monomer further include ester and salts of the monomers. Inorganic materials include but are not limited to silica.

Thickening Agent—The composition of the present invention may include one or more thickening agents. The composition of the present invention may comprise from about 0.1% to about 5%, or, alternatively, from about 0.2% to about 2%, of a thickening agent when present. Suitable classes of thickening agents include but are not limited to carboxylic acid polymers, polyacrylamide polymers, sulfonated polymers, copolymers thereof, hydrophobically modified derivatives thereof, and mixtures thereof.

Suitable thickening agents include carboxylic acid polymers such as the carbomers (e.g., the CARBOPOL® 900 series such as CARBOPOL® 954). Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic Corp. and Viscolam At 100 P available from Lamberti S.p.A. (Gallarate, Italy). Another commercially available material comprising a sulfonated polymer is Sepiplus 400 available from Seppic Corp.

Humectants—The composition of the present invention may include one or more humectants. The composition of the present invention may comprise from about 3% to about 25%; alternatively, from about 5% to about 15%; or, alternately, from about 7% to about 12% of the humectant, when present. An exemplary class of humectants is polyhydric alcohols. Suitable polyhydric alcohols include polyalkylene glycols and alkylene polyols and their derivatives, including propylene glycol, dipropylene glycol, polypropylene glycol, polyethylene glycol and derivatives thereof; sorbitol; hydroxypropyl sorbitol; erythritol; threitol; pentaerythritol; xylitol; glucitol; mannitol; hexylene glycol; butylene glycol (e.g., 1,3-butylene glycol); pentylene glycol; hexane triol (e.g., 1,2,6-hexanetriol); glycerine; ethoxylated glycerine; and propoxylated glycerine.

Other suitable humectants include sodium 2-pyrrolidone-5-carboxylate, guanidine; glycolic acid and glycolate salts (e.g., ammonium and quaternary alkyl ammonium); lactic acid and lactate salts (e.g., ammonium and quaternary alkyl ammonium); aloe vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; panthenol; sodium pyroglutamate (NaPCA), water-soluble glyceryl poly(meth)acrylate lubricants (such as Hispagel®) and mixtures thereof.

Opacifying Particulate Material—In certain embodiments, the personal care composition comprises no more than about 5% of opacifying particulate material. In other embodiments, the personal care composition comprises no more than about 3% or about 1% of opacifying particulate material. In certain embodiments of the present invention, the personal care composition is substantially devoid (i.e., less than 0.01%) of opacifying particulate material. An “opacifying particulate material” refers to a non-fluid material having a refractive index of greater than 1.9. Refractive index can be determined by conventional methods. While not wishing to be bound in theory, limiting or eliminating the amount opacifying particulate material provides a personal care composition that achieves a visible acute and/or chronic benefit by a mechanism other than by merely “masking” the skin or other keratinous tissue. In other words, the resulting personal care composition does not act as a make-up that is traditionally designed to merely conceal signs of skin aging and other skin imperfections.

Examples of opacifying particulate materials are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), under the chemical class of “Opacifying Agents.” Exemplary opacifying particulate materials include titanium dioxide, zinc oxide, zirconium dioxide, iron oxides, pigments, and the like.

Actives—The composition of the present invention may comprise at least one skin care active (“active”), useful for regulating and/or improving the condition of mammalian skin. The active may be soluble in oil or water, and may be present primarily in the oil phase and/or in the aqueous phase. Solubility in water and oil is within the knowledge of one of skill in the art, and can be determined using known methods of analysis. One of skill in the art further will understand that solubility may be affected by the type and concentration of other components in the composition, and other conditions such as pH, ionic strength, etc. Many skin care actives may provide more than one benefit, or operate via more than one mode of action; therefore, classifications herein are made for the sake of convenience and are not intended to limit the active to that particular application or applications listed. Suitable actives include, but are not limited to, vitamins, peptides, sugar amines, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals.

The compositions of the present invention may comprise from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®). The compositions may comprise from about 1×10⁻⁷% to about 20%, alternatively from about 1×10⁻⁶% to about 10%, and alternatively from about 1×10⁻⁵% to about 5% of the peptide.

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

The compositions of the subject invention may comprise one or more sunscreen actives (or sunscreen agents) and/or ultraviolet light absorbers. Herein, suitable sunscreen actives include oil-soluble sunscreens, insoluble sunscreens, and water-soluble sunscreens. In certain embodiments, the composition may comprise from about 1% to about 20%, or, alternatively, from about 2% to about 10%, by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof.

Non-limiting examples of suitable insoluble sunscreens include methylene bis-benzotriazolyl tetramethylbutyl-phenol, titanium dioxide, zinc cerium oxide, zinc oxide, and derivatives and mixtures thereof.

Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquarternium-59, TEA-salicylate, and salts, derivatives and mixtures thereof.

The compositions of the present invention may comprise one or more compounds for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

The compositions of the present invention may comprise a tanning active. The compositions may comprise from about 0.1% to about 20%, from about 2% to about 7%, or, alternatively, from about 3% to about 6%, by weight of the composition, of a tanning active. A suitable tanning active includes dihydroxyacetone, which is also known as DHA or 1,3-dihydroxy-2-propanone.

The compositions of the present invention may comprise a safe and effective amount of one or more anti-acne actives. Examples of useful anti-acne actives include resorcinol, sulfur, salicylic acid, erythromycin, zinc, etc. Suitable anti-acne actives are described in further detail in U.S. Pat. No. 5,607,980.

The compositions of the present invention may comprise a safe and effective amount of a desquamation active such as from about 0.01% to about 10%, from about 0.5% to about 5%, or, alternatively, from about 0.1% to about 2%, by weight of the composition. Desquamation actives enhance the skin appearance benefits of the present invention. For example, the desquamation actives tend to improve the texture of the skin (e.g., smoothness). A suitable desquamation system comprises sulfhydryl compounds and zwitterionic surfactants and is described in U.S. Pat. No. 5,681,852. Another suitable desquamation system comprises salicylic acid and zwitterionic surfactants and is described in U.S. Pat. No. 5,652,228.

The compositions of the present invention may comprise a safe and effective amount of an anti-cellulite agent. Suitable agents may include, but are not limited to, xanthine compounds (e.g., caffeine, theophylline, theobromine, and aminophylline).

The compositions of the present invention may comprise a safe and effective amount of a chelating agent such as from about 0.1% to about 10% or from about 1% to about 5% of the composition. Exemplary chelators are disclosed in U.S. Pat. No. 5,487,884; International Publication No. WO91/16035; and International Publication No. WO91/16034. A suitable chelator is furildioxime and derivatives.

The compositions of the present invention may comprise a skin lightening agent. The compositions may comprise from about 0.1% to about 10%, from about 0.2% to about 5%, or, alternatively, from about 0.5% to about 2%, by weight of the composition, of a skin lightening agent. Suitable skin lightening agents include kojic acid, arbutin, tranexamic acid, ascorbic acid and derivatives (e.g., magnesium ascorbyl phosphate or sodium ascorbyl phosphate or other salts of ascorbyl phosphate), ascorbyl glucoside, and the like. Other suitable skin lightening materials include undecylenoyl phenylalanine (Sepiwhite® from SEPPIC), aloesin, Actiwhite® (Cognis), and Emblica® (Rona).

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773.

The compositions of the present invention may comprise protease inhibitors including, but are not limited to, hexamidine (including salts and derivates thereof), vanillin acetate, menthyl anthranilate, soybean trypsin inhibitor, Bowman-Birk inhibitor, and mixtures thereof.

The compositions of the present invention may other optional components such as non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobial or antifungal actives, and other useful skin care actives, which are described in further detail in U.S. application publication No. US2006/0275237A1 and US2004/0175347A1.

### II. Methods of Use

The present invention describes a method of regulating and/or improving the condition of mammalian skin. The method comprises the step of topically applying to mammalian skin a personal care composition described herein. The composition may be applied to any keratinous tissue, including keratinous tissue in need of one or more benefits. In one embodiment, the method may comprise the step of applying the composition described herein to insult-affected keratinous tissue, to regulate and/or improve the condition of such tissue, and/or to provide relief from the effects of the insult.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices.

### III. Consumer Product

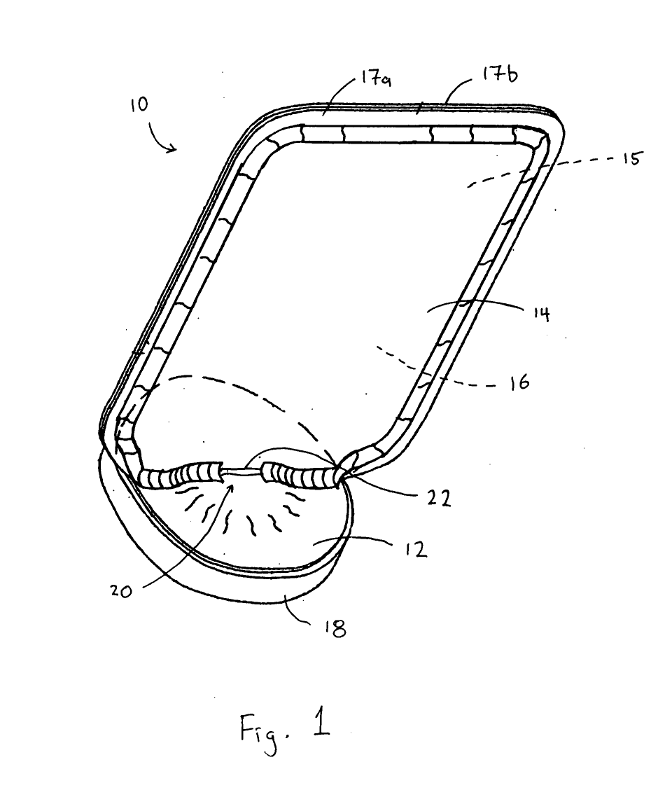

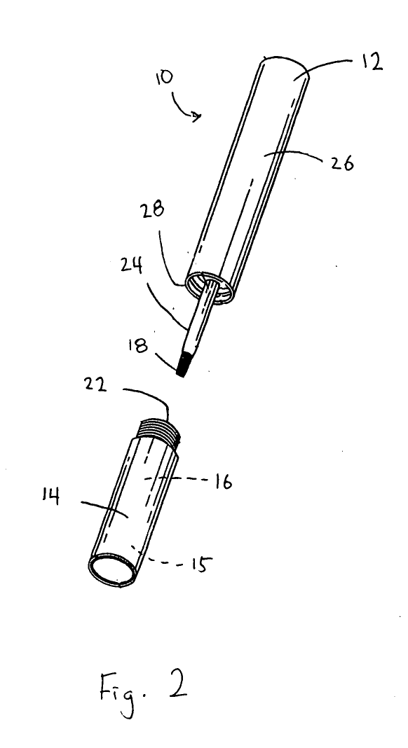

The present invention further relates to a consumer product that contains the personal care composition. FIGS. 1-2 depict several suitable and exemplary embodiments of the consumer product (10). It should be readily recognized that the consumer product (10) is not limited to the particular embodiments shown. Specifically, the consumer product (10) may take any suitable size or shape. The consumer product (10) may comprise an applicator (12) and a reservoir (14) with the personal care composition (16) disposed therein. The applicator (12) is the portion of the consumer product (10) that delivers the personal care composition to the mammalian keratinous tissue (e.g., skin). In certain embodiments, the applicator (12) may comprise a substrate (18) that contacts the skin and transfers the personal care composition to the skin. The substrate (18) has a skin-contacting surface that, in use, is in physical contact with the skin. The substrate (18) may comprise any material that is non-irritating to the skin. Suitable substrates include nonwoven webs (e.g., spunbond, meltblown, carded, and other conventionally formed webs), woven fabrics, foam, fibrous wadding, tissue, paper, a plurality of fibers or bristles, and the like. The substrate (18) may be able to deliver a quantity of the personal care composition (16). Suitable substrates (18) may be porous. In certain embodiments, the skin-contacting surface of the substrate (18) has a surface area of less than about 6.5 cm². The relatively small surface area facilitates the use of the consumer product (10) as a spot-treatment device. A spot-treatment device allows for application of the consumer product (10) to a limited area of skin needing regulation or improvement. For example, a sign of skin aging is the appearance of wrinkles in the temple region adjacent the eyes or adjacent the edges of the mouth (i.e., often referred to a “crow's feet”). The small surface area of the substrate (18) allows for targeted application of the personal care composition (16) to the skin area of concern. Such application is also helpful when applying the personal care composition (16) to insult-affected keratinous tissue thereby targeting the areas of insult.

The reservoir (14) has a void (15) that may have a quantity of the personal care composition (16) disposed therein. The reservoir (14) may be in any suitable form such as ajar, tube, packet, pouch, vial, or any other form capable of retaining a quantity of the personal care composition (16) until application. The reservoir (14) may be constructed to contain a quantity of the personal care composition (16) permitting a single-use or multiple-use of the consumer product (10). In a single-use consumer product (10), the reservoir (14) may be constructed with such that the void (15) has a volume sufficient to contain a quantity of the personal care composition (16) for a single-use after which the consumer product is substantially devoid of the personal care composition (16). A suitable reservoir (14) for a single-use product may have void (15) with a volume of less than about 2 cm³ or, alternatively, less than about 1 cm³. In a multiple-use consumer product (10), the reservoir (14) may be constructed with a void volume sufficient to contain a quantity of the personal care composition (16) for a several uses. The reservoir (14) of a multiple-use consumer product (10) may replenish the applicator (12) with personal care composition (16) after each application. In certain embodiments, the reservoir (14) may be partially or fully translucent. As used herein, “translucent” includes transparent. A translucent reservoir (14) allows a user to inspect the personal care composition (16) within the void (15). The translucency provides a mechanism in which to communicate to the user that the personal care composition (16), in some embodiments, has a low percentage of to substantially no opacifying particulate material. In certain embodiments, the reservoir (14) comprises an opaque region and a translucent region.

The substrate (18) is in liquid communication with the reservoir (14) such that the personal care composition (16) is transmitted from the reservoir (14) to the substrate (18). However, the substrate (18) need not maintain liquid communication with the reservoir (14) during the normal use of the consumer product (10). The reservoir (14) may be integral to or detachable from the applicator (12). An integral reservoir (14) remains in liquid communication to the substrate (18) during the normal use of the consumer product (10). A detachable reservoir (14) may lose liquid communication with the substrate (18) during normal use of the consumer product (10).

FIG. 1 depicts the consumer product (10) as an integral product, wherein the applicator (12) and the reservoir (14) are not separated during normal use of the product. The reservoir (14) is a pouch formed from two liquid impermeable layers (17a, 17b) that are bonded to form a void (15) there between that contains the personal care composition (16). The applicator (12) comprises a portion of the two liquid impermeable layers (17a, 17b) joined to the substrate (18) such as an open-cell foam sheet. The reservoir (14) is tapered to form a conduit (20) to the applicator (12). The personal care composition (16) may be conveyed from the reservoir (14) through the conduit (20) to the substrate (18) of the applicator (12). The conduit (20) may contain a frangible seal (22) that prevents inadvertent conveyance of the personal care composition (16) from the reservoir (14) to the substrate (18). The frangible seal (22) may be a thin plastic film, a wax plug, a line of light bonding between the two liquid impermeable layers, or any other conventional means of frangibly sealing the conduit (20). To use the consumer product (10), a user may break the frangible seal (22) by applying pressure to the reservoir (14) or by other suitable means as dictated by the frangible seal (22). The personal care composition (16) flows from the reservoir (14) through the conduit (20) to the substrate (18) of the applicator (12). The user may grasp the reservoir (14) which may serve as a handle during application of the personal care composition (16) to the skin of the user by contacting the substrate (18) to the skin.

FIG. 2 depicts the consumer product (10) having a detachable reservoir (14), wherein the applicator (12) and the reservoir (14) are separated from liquid communication during normal use of the product. The reservoir (14) is a vial made from any suitable rigid, liquid impermeable material (e.g., plastic, glass, metal, etc.) with a void (15) therein. The personal care composition (16) is contained within the void (15) of the reservoir (14). The reservoir (14) is shown having a threaded member (22) adjacent the open end of the reservoir (14). The applicator (12) contains a main body (26) that may be used as a handle during application of the personal care composition (16). The main body (26) may have a threaded member (28) complementary to the threaded member (22) of the reservoir (14). The applicator (12) also comprises a stem (24) which is joined the main body (26). The substrate (18) is joined to the stem (24). When the consumer product (10) is not in use, the threaded member (28) of the main body (26) may be engaged with the threaded member (22) of the reservoir (14). In this closed configuration, the substrate (18) is in liquid communication with the personal care composition (16). The closed configuration allows the substrate (18) to be impregnated or coated with the personal care composition (16).

During use of the consumer product (10), the applicator (12) and the reservoir (14) are separated (i.e., the threaded members 22, 24 are disengaged). The applicator (14) (specifically, the stem (24) and substrate (18)) are removed from the void (15) of the reservoir (14). The substrate (18) having a quantity of the personal care composition (16) disposed thereon or therein is applied to the skin of a user by contacting the substrate (18) to the skin.

The amount of the composition applied, the frequency of application, and the period of use will vary widely depending upon the level of components of a given composition and the level of regulation desired. In one embodiment, the composition is applied at least once daily, where “daily” mean a 24-hour period. The composition further may be applied as part of a treatment regimen, for example, once daily for 30 consecutive days, for 14 consecutive days, for 7 consecutive days, or, alternatively, for 2 consecutive days.

### IV. Examples

| | Example 1 | Example 2 | Example 3 | Example 4 | Example 5 |
|---|---|---|---|---|---|
| Water Phase: | | | | | |
| Water | qs to 100% | qs to 100% | qs to 100% | qs to 100% | qs to 100% |
| Glycerin | 11 | 4 | 10 | 5 | 7 |
| Butylene Glycol | — | — | 1 | — | — |
| Propylene Glycol | — | — | 1 | — | — |
| Disodium EDTA | 0.05 | 0.05 | 0.05 | 0.02 | 0.05 |
| Niacinamide | 5 | 4 | 5 | 2 | 3.5 |
| D-panthenol | 0.7 | 1 | 1 | 0.25 | 0.5 |
| N-acetyl glucosamine | 2 | 2 | — | — | 0.5 |
| Matrixyl¹ | — | — | 0.353 | 0.26 | — |
| Polysorbate 20 | 0.6 | 0.8 | 1 | 0.7 | 0.8 |
| Glydant Plus Liquid² | 0.3 | 0.3 | — | 0.2 | 0.3 |
| Triethanolamine | — | 0.09 | — | — | — |
| Allantoin | — | — | 0.1 | — | — |
| Caffeine | — | — | 1 | — | — |
| Green Tea Extract | — | — | 1 | 0.5 | — |
| Cucumber Extract | — | — | 1.5 | — | — |
| Witch Hazel Extract | — | — | 0.5 | — | — |
| Aloe Vera | — | — | 0.15 | 0.1 | — |
| Benzyl alcohol | — | — | 0.4 | — | — |
| Silicone/Oil Phase: | | | | | |
| Cyclomethicone D5 | 15 | 16 | 18 | 10 | 15 |
| Dow Corning 9045³ | 15 | 20 | 13 | — | — |
| Dow Corning 9040⁴ | — | — | — | 10 | 20 |
| KSG-15⁵ | — | — | — | 10 | — |
| Dow Corning 1503⁶ | 3 | 4 | 3.5 | — | 2 |
| Dimethicone 50 csk | 6 | 5 | 5 | 3 | — |
| Vitamin E Acetate | 0.2 | 0.5 | 0.5 | 0.1 | 0.5 |
| Isopropyl lauroyl | — | 2 | — | — | — |
| Sarcosinate | | | | | |
| Undecylenoyl | — | 0.2 | — | — | — |
| Phenylalanine | | | | | |
| Laureth-4 | 0.2 | 0.2 | 0.2 | 0.2 | 0.2 |
| Ethylparaben | — | — | 0.1 | — | — |
| Propylparaben | — | — | 0.1 | — | — |
| Thickener: | | | | | |
| Sepigel 305⁷ | 1.6 | 3.0 | 1.6 | — | — |
| Simulgel EG⁸ | — | — | — | 2 | 3 |
| Additional Ingredients: | | | | | |
| Microthene FN510-00⁹ | 9 | 9.5 | 8 | — | — |
| Tospearl 145A¹⁰ | — | — | — | — | 8 |
| Dry Flo Pure¹¹ | — | — | — | 9.5 | — |
| Prestige Silk Red¹² | — | — | — | — | 1.0 |
| GLW75CAP-MP¹³ | — | — | 0.35 | — | — |
| Total: | 100% | 100% | 100% | 100% | 100% |

¹Palmitoyl-pentapeptide-4 blend available from Sederma
²DMDM Hydantoin and Iodopropynyl Butylcarbamate blend available from Lonza, Inc.
³Dimethicone Crosspolymer in Cyclomethicone from Dow Corning Corporation
⁴Dimethicone Crosspolymer in Cyclomethicone from Dow Corning Corporation
⁵Dimethicone/Vinyl Dimethicone Crosspolymer in Cyclomethicone from Shin-Etsu Chemical Co.
⁶Dimethicone/Dimethiconol blend from Dow Corning Corporation
⁷Polyacrylamde, C13–14 Isoparaffin, and Laureth-7 from Seppic
⁸Sodium Acrylate/Sodium Acryloyldimethyl Taurate Copolymer, Isohexadecane, and Polysorbate 80 from Seppic
⁹Polyethylene Homopolymer particles from Equistar
¹⁰Polymethylsilsesquioxane particles from GE Toshiba Silicone Company Ltd.
¹¹Aluminum Starch Octenylsuccinate particles from National Starch and Chemical Company
¹²Titanium dioxide, mica, and tin oxide from Eckart
¹³75% aqueous titanium dioxide dispersion from Kobo Products, Inc.

In a suitable vessel, the water phase ingredients are combined and mixed until uniform; the water phase may be warmed to dissolve all ingredients. In a separate suitable container, the silicone/oil phase ingredients are combined and mixed until uniform; the silicone/oil phase may be warmed to dissolve all ingredients. Half the thickener and then the silicone/oil phase is added to the water phase and the resulting emulsion is milled (e.g., with a Tekmar T-25). The remainder of the thickener and then the remaining ingredients are then added to the emulsion while stirring. Once the composition is uniform, the product is poured into suitable containers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition in the form of a oil-in-water emulsion comprising:
a) from about 0.5% to about 10% of a silicone elastomer;
b) from about 20% to about 50% of a silicone fluid;
c) from about 5% to about 30% of a particulate material having a refractive index of less than about 1.8;
d) from about 0.2% to about 2% of a polymeric thickener; and
e) water.

2. The composition of claim 1 wherein the composition comprises from about 1% to about 5% of the silicone elastomer.

3. The composition of claim 1 wherein the silicone elastomer is a non-emulsifying silicone elastomer.

4. The composition of claim 1 wherein the composition comprises from about 25% to about 40% of the silicone fluid.

5. The composition of claim 1 wherein the silicone fluid is a volatile silicone.

6. The composition of claim 1 wherein the silicone fluid is selected from a group consisting of polyalkylsiloxanes, cyclomethicones, and combinations thereof.

7. The composition of claim 1 wherein the composition comprises from about 6% to about 15% of the particulate material.

8. The composition of claim 1 wherein the particulate material has a median particle size of from about 0.1 μm to about 40 μm.

9. The composition of claim 1 wherein the particulate material has a median particle size of from about 3 μm to about 20 μm.

10. The composition of claim 1 wherein the particulate material is selected from a group consisting of polymethylsilsesquioxanes, siloxanes, polyamides, polyolefins, polystyrenes, polytetrafluoroethylenes, polyurethanes, polymethacrylates, starch and starch derivatives, composite particles, copolymers thereof, and combinations thereof.

11. The composition of claim 1 wherein the composition comprises from about 0.4% to about 1.5% of a polymeric thickener.

12. The composition of claim 1 wherein the polymeric thickener is selected from a group consisting of carboxylic acid polymers, polyacrylamide polymers, sulfonated polymers, copolymers thereof, hydrophobically modified derivatives thereof, and mixtures thereof.

13. The composition of claim 12 wherein the polymeric thickener is selected from a group consisting of polyacrylamide polymers, sulfonated polymers, copolymers thereof, and mixtures thereof

14. The composition of claim 1 wherein the composition further comprises from about 3% to about 25% of a humectant.

15. The composition of claim 14 wherein the composition comprises from about 5% to about 15% of a humectant.

16. The composition of claim 14 wherein the humectant is low molecular weight polyol.

17. The composition of claim 16 wherein said low molecular weight polyol is glycerine.

18. The composition of claim 1 wherein the composition further comprises a skin active selected from a group consisting of vitamins, peptides, sugar amines, sunscreens, oil control agents, non-vitamin antioxidants and radical scavengers, hair growth regulators, flavonoids, minerals, phytosterols, plant hormones, protease inhibitors, tyrosinase inhibitors, anti-inflammatory agents, N-acyl amino acid compounds, and combinations thereof.

19. The composition of claim 18 wherein the skin active is a vitamin B compound, a vitamin C compound, N-acetyl glucosamine, hexamidine, derivatives thereof, or combinations thereof.

20. The composition of claim 1 wherein said composition comprises no more than about 5% of an opacifying particulate material.

21. The composition of claim 1 wherein said composition comprises no more than about 1% of an opacifying particulate material.

22. The composition of claim 1 wherein said composition is substantially devoid of an opacifying particulate material.

23. The composition of claim 1 wherein the composition comprises from about 8% to about 74% of water.

24. The composition of claim 1 wherein the composition comprise from about 35% to about 60% of water.

25. A consumer product comprising
a) an applicator comprising a substrate, wherein the substrate has a skin-contacting surface;
b) a reservoir containing a quantity of the personal care composition according to claim 1, wherein the reservoir is in liquid communication to the substrate.

26. The consumer product of claim 25 wherein the skin-contacting surface of the substrate has a surface area of less than about 6.5 cm².

27. The consumer product of claim 25 wherein the reservoir has an open volume of less than about 1 cm³.

28. The consumer product of claim 25 wherein the applicator and reservoir are integral.

29. The consumer product of claim 25 wherein the reservoir comprises a translucent region.
